(19) United States
(12) Patent Application Publication
(10) Pub. No.: US 2026/0334947 A1
(43) Pub. Date: Oct. 29, 2026

(54) Cartridge For Surgical Staples

(71) Applicant: PETERS SURGICAL, BOULOGNE BILLANCOURT (FR)

(72) Inventor: Denis DEPERNE, BOULOGNE BILLANCOURT (FR)

(21) Appl. No.: 19/107,900

(22) PCT Filed: Aug. 3, 2023
(86) PCT No.: PCT/FR2023/051236

§ 371 Date: Feb. 28, 2025

(30) Foreign Application Priority Data
Sep. 2, 2022 (FR) FR2208836

(51) Int. Cl. A61B 17/072

(52) U.S. Cl. CPC A61B 17/072; A61B 2017/07271

(57) ABSTRACT

The invention relates to a cartridge (1) comprising housings (2, 3, 4) for storing respective surgical staples, each of the surgical staples comprising two arms, one of the two arms being rotatably movable about an axis of rotation relative to the other arm in order to close the surgical staple, wherein all the housings (2, 3, 4) of the cartridge are designed so that, when the surgical staples are respectively stored in all the housings (2, 3, 4) of the cartridge (1), the surgical staples adopt respective positions such that a plane (P) perpendicular to the axis of rotation (R) of each of the surgical staples intersects each of the surgical staples. (

### FIELD OF THE INVENTION

The present invention relates to a cartridge for surgical staples, also referred to in the literature as surgical “clips”.

### STATE OF THE ART

A conventional surgical staple comprises two arms, one of which is rotatably movable about an axis of rotation relative to the other arm to close the surgical staple. Such a surgical staple is typically used to be placed within a patient's body. For example, the surgical staple may be used to perform a ligation, i.e. to bring disjointed biological tissues together to stop bleeding.

Document US 2019/0133594 describes a cartridge comprising a row of housings for storing surgical staples of this type. The row of housings is designed so that, when the surgical staples are stored in the row of housings, the surgical staples adopt respective positions such that the axes of rotation of the surgical staples are coincident.

Such a cartridge thus forms a block having a length, a width and a height which are relatively balanced relative to each other.

A user removes the staples stored in the cartridge one by one using an instrument, in order to place them on or in a patient's body.

However, such a cartridge proves cumbersome in certain situations.

For example, it may be necessary to place several surgical staple cartridges in a pouch that has a substantially flat shape. Filling a pouch with cartridges having the shape described in US 2019/0133594 is not optimal. This is because the two walls of a pouch can only deform to a certain extent, leaving substantial void areas therebetween once cartridges have been inserted into the pouch. Moreover, it is not possible to fill these void areas without compromising the closure of the pouch.

Moreover, it has been proposed to temporarily insert a cartridge inside a patient's body, in the case where staples are to be placed at a site inside the patient's body. In this case, the cartridge is typically inserted into the patient's body via a first trocar, and an instrument for retrieving the staples is inserted into the body via a second trocar, the retrieval of the staples from the cartridge and their delivery to the placement site being carried out in the body, by means of this instrument. This method avoids the user having to go back and forth between the inside and outside of the body to place different staples and thus reduces the duration of the operation. However, the cartridge described in the document with cartridges having the shape described in document US 2019/0133594 is not well suited to such temporary intracorporeal insertion, due to its bulk.

Another solution to avoid going back and forth between the inside and outside of the patient's body is to use an applicator in which the staples are pre-loaded. However, this applicator must necessarily comprise a complex mechanism to ensure the functions of storing and placing the staples. The manufacturing cost of this applicator is therefore high.

### DISCLOSURE OF THE INVENTION

A goal of the invention is to propose a cartridge more suited to certain uses, in particular packaging in a pouch and intracorporeal insertion.

This purpose is achieved by a cartridge comprising housings for storing respective surgical staples, each of the surgical staples comprising two arms, one of the two arms being rotatably movable about an axis of rotation relative to the other arm in order to close the surgical staple, wherein all the housings of the cartridge are designed so that, when the surgical staples are respectively stored in all the housings of the cartridge, the surgical staples adopt respective positions such that a plane perpendicular to the axis of rotation of each of the surgical staples intersects each of the surgical staples.

By orienting the housings in this way, the dimension of the cartridge, measured in a direction normal to the plane intersecting the staples, can be greatly reduced compared to the other two dimensions of the cartridge measured parallel to this plane. The proposed cartridge can thus have a much more flattened shape than the cartridge proposed in document US 2019/0133594. Cartridges of this flattened shape can be inserted into a bag in greater numbers than cartridges having the shape proposed in document US 2019/0133594.

Another advantage of the cartridge is that it can be more easily grasped by two jaws of a surgical clamp. This type of instrument is routinely available in an operating room.

Yet another advantage of the cartridge is that it can more easily be inserted into a trocar, which has an elongated shape. The cartridge can therefore be more easily inserted into a patient's body than the cartridge proposed in document US 2019/0133594, for the purpose of extracting staples from the cartridge carried out in the body.

The cartridge described above constitutes a first aspect of the present disclosure.

The cartridge according to this first aspect may also comprise the following features, taken alone or in combination whenever technically possible.

Preferably, the cartridge comprises the surgical staples respectively stored in all the housings.

Preferably, the housings comprise a first row of housings opening onto a first side of the cartridge, and a second row of housings opening onto a second side of the cartridge opposite the first side.

Preferably, the cartridge comprises:
a first wall and a second wall delimiting the housings therebetween, the first wall having a first outer surface and the second wall having a second outer surface opposite the first outer surface,
two stops projecting from the first outer surface so as to delimit a recess therebetween, the two stops being arranged to allow the cartridge to be placed in a position gripped by an instrument, in which:
   a first jaw of the instrument bears on the first outer surface and a second jaw of the instrument bears on the second outer surface,
   each housing of the cartridge is accessible via a first side of the instrument or via a second side of the instrument opposite the first side,
   the first jaw extends into the recess between the two stops.

Preferably, the cartridge comprises two second stops projecting from the second outer surface so as to delimit therebetween a second recess, the two second stops being arranged so that, in the gripped position, the second jaw extends into the second recess between the second stops.

Preferably, the cartridge comprises first blocking means for limiting movement of the cartridge relative to a first adjacent structure in a direction parallel to the plane, the first blocking means comprising at least one of:
a first male element adapted to be engaged with a female element of the adjacent first structure,
a first female element adapted to be engaged with a male element of the adjacent first structure.

Preferably, the first blocking means are adapted to limit a translational movement of the cartridge relative to the first adjacent structure in any direction parallel to the plane.

Preferably, the cartridge comprises a first wall having a first planar surface, wherein the first male element projects from the first planar surface and has a first slope extending the first planar surface, the first slope being inclined relative to the first planar surface at an obtuse angle.

Preferably, the cartridge comprises second blocking means for limiting movement of the cartridge relative to a second adjacent structure in a direction parallel to the plane, the second blocking means being opposite the first blocking means relative to the housings, the second blocking means comprising at least one of:
a second male element adapted to be engaged with a female element of the second adjacent structure,
a second female element adapted to be engaged with a male element of the second adjacent structure.

Preferably, the second blocking means are adapted to limit a translational movement of the cartridge relative to the second adjacent structure in any direction parallel to the plane.

Preferably, the second blocking means are symmetrical to the first blocking means relative to an axis.

Also provided, according to a second aspect, is a set of cartridges comprising:
a first cartridge according to the first aspect,
a second cartridge according to the first aspect
in which the first blocking means of the second cartridge are adapted to:
   be engaged with the first blocking means of the first cartridge, when the first cartridge is in a first orientation relative to the second cartridge, and
   be engaged with the second blocking means of the first cartridge, when the first cartridge is in a second orientation relative to the second cartridge, the second orientation being different from the first orientation.

Also provided, according to a third aspect, is a system comprising:
a set of cartridges comprising a first cartridge and a second cartridge according to the first aspect,
a case comprising:
   a first flap extending in a first plane and a second flap extending in a second plane, the first flap 52 and the second flap 54 defining therebetween a space for storing the set of cartridges in a stacked position in which the plane of the first cartridge and the plane of the second cartridge are parallel to the first plane and the second plane.

The system according to this third aspect may also comprise the following features, taken alone or in combination whenever technically possible.

Preferably, the first flap is movable, when the set of cartridges is stored in the space in the stacked position, between:
a first position in which the first flap is engaged with the first cartridge, so as to limit a translational movement of the first cartridge relative to the first flap in a direction parallel to the first plane,
a second position, in which the first flap is not engaged with the first cartridge.

Preferably:
the case defines an access to the space allowing insertion of the set of cartridges into the space,
the first slope is arranged so that, during insertion of the set of cartridges into the space via the access, the first flap bears on the slope, and sliding of the first flap on the slope moves the first flap from the first position to the second position.

Preferably:
the first flap comprises a female element,
the first male element of the first cartridge engages with the female element of the first flap in the first position.

Preferably, the second flap is movable, when the set of cartridges is stored in the space in the stacked position, between:
a first position in which the second flap is engaged with the second cartridge, so as to limit a translational movement of the second cartridge relative to the second flap in a direction parallel to the second plane,
a second position, in which the second flap is not engaged with the second cartridge.

Preferably:
the case defines an access to the space allowing insertion of the set of cartridges into the space,
The case comprises a base comprising:
   a central portion forming a bottom of the space opposite the access,
   a first peripheral portion extending the central portion 76 beyond the first flap, and optionally a second peripheral portion extending the central portion beyond the second flap, the second peripheral portion being opposite the first peripheral portion relative to the central portion.

Preferably:
the case defines an access to the space allowing the set of cartridges to be inserted into the space,
The case comprises:
   a base forming a bottom of the space opposite the access, the first flap being connected to the base by a first fold line, and the second flap being connected to the base by a second fold line,
   a connecting flap connected to the first flap by a third fold line perpendicular to the first fold line, and moreover connected to the second flap by a fourth fold line perpendicular to the second fold line.

Preferably, the case is made of cardboard.

### DESCRIPTION OF THE FIGURES

Other features, aims and advantages of the invention will emerge from the following description, which is purely illustrative and non-limiting, and which must be read in conjunction with the appended drawings in which:

FIG. 1 is a perspective view of a surgical staple cartridge, according to one embodiment.

FIG. 2 is another perspective view of the cartridge shown in FIG. 1.

FIG. 3 is a sectional view of the cartridge shown in FIG. 1.

FIG. 4 is a top view of the cartridge shown in FIG. 1.

FIG. 5 is a top view of a set of cartridges according to a first embodiment.

FIG. 6 is a perspective view of a set of cartridges according to a second embodiment, and a case according to one embodiment.

FIG. 7 is a perspective view of the set of cartridges and the case of FIG. 6 arranged within each other.

FIG. 8 is a perspective view of a stack of cases, according to one embodiment.

FIG. 9 is a perspective view of a package containing a plurality of cartridges, according to one embodiment.

FIG. 10 is a sectional view of a cartridge according to a second embodiment.

FIG. 11 is a perspective view of the cartridge according to the second embodiment.

Throughout the figures, similar elements have identical references.

### DETAILED DESCRIPTION OF THE INVENTION

### 1) Cartridge for Surgical Staples

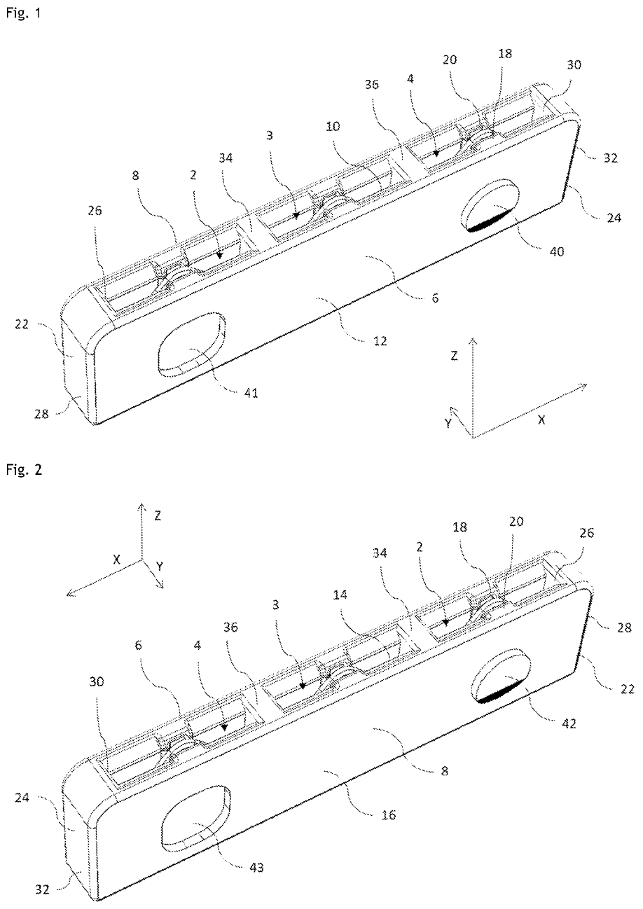

FIGS. 1 to 4 show a cartridge 1 according to a first embodiment, for storing a plurality of surgical staples.

Cartridge 1 extends in a longitudinal direction indicated by an axis X, in a transverse direction indicated by an axis Y, and in a vertical direction indicated by an axis Z, the axes X, Y and Z forming an orthogonal reference frame.

Generally, cartridge 1 has an elongated shape in the longitudinal direction, in the sense that its length measured parallel to the axis X is greater than its width measured parallel to the axis Y and its height measured parallel to the axis Z.

Cartridge 1 comprises N housings 2, 3, 4 to store N surgical staples (one staple per housing). The N housings form a single row in the longitudinal direction. In other words, when traveling along the longitudinal direction, each housing in the row is traveled successively.

The single row of housings comprises two extreme housings 2, 4 (that is to say at the end of the row). The single row of housings may also comprise at least one intermediate housing (that is to say at least one housing between two other housings in the row). In the embodiment shown in FIG. 1, N=3, which implies that there is only one intermediate housing 3 in cartridge 1 between the two extreme housings 2, 4 of the row. However, it is understood that N may be equal to 2 or strictly greater than 3.

Cartridge 1 comprises a first wall 6 and a second wall 8 between which the single row of housings, 2, 3, 4, is arranged.

The first wall 6 has a first inner surface 10, delimiting each of the housings 2, 3, 4 of the single row, and a first outer surface 12 opposite the first inner surface 10. The first outer surface 12 forms a first edge of the cartridge 1. The first inner surface 10 and the first outer surface 12 extend in the longitudinal direction and in the vertical direction. The first outer surface 12 is planar and extends in a first plane parallel to the plane (X, Z).

With reference to FIG. 2, the second wall 8 has a second inner surface 14, delimiting each of the housings of the single row, and a second outer surface 16 opposite the second inner surface 14. The second outer surface 16 forms a second edge of the cartridge 1 opposite the first edge. The second inner surface 14 and the second outer surface 16 extend in the longitudinal direction. The second outer surface 16 is planar and extends in a second plane parallel to the plane (X, Z).

The first wall 6 and the second wall 8 comprise portions 18, 20 that are deformable by compression, serving as support points for the surgical staples when said staples are stored in the housings. The deformable portions 18, 20 are arranged to be facing each other in each of the housings 2, 3, 4, such that a staple stored in any of the housings 2, 3, 4 presses simultaneously on the deformable portions 18, 20 to compress them, which allows the staple to be held by compression in the housing. The deformable portions 18, 20 are for example ribs projecting from the first inner surface 10 and from the second inner surface 14. Ultimately, the deformable portions 18, 20 therefore form retaining means for retaining surgical staples in the housings 2, 3, 4. However, it is understood that this retaining function can be ensured by retaining means having a different shape.

The cartridge 1 further comprises a third wall 22 and a fourth wall 24 between which the single row of housings 2, 3, 4 is arranged. The third wall 22 and the fourth wall 24 each connect the first wall 6 to the second wall 8.

The third wall 22 has a third inner surface 26, delimiting only the end housing 2, and a third outer surface 28 opposite the third inner surface 26. The third outer surface 28 forms a third edge of the cartridge 1. The third inner surface 26 and the third outer surface 28 extend in the transverse direction, as well as in the vertical direction. The third outer surface 28 is perpendicular to the first outer surface 12 and to the second outer surface 16.

The fourth wall 24 has a fourth inner surface 30, delimiting only the other end housing of the single row, and a fourth outer surface 32 opposite the fourth inner surface 30. The fourth outer surface 32 forms a fourth edge of the cartridge 1 opposite the third edge. The fourth inner surface 30 and the fourth outer surface 32 extend in the transverse direction, as well as in the vertical direction. The fourth outer surface 32 is perpendicular to the first outer surface 12 and to the second outer surface 16.

Cartridge 1 moreover comprises a plurality of partitions 34, 36. Each partition 34, 36 connects the first wall 6 to the second wall 8, extending from the first inner surface 10 to the second inner surface 14. Each partition 34, 36 separates two adjacent housings of the single row. The number of partitions is equal to N−1, such that each housing can only store one surgical staple at a time. Thus, in the embodiment illustrated in FIGS. 1 to 4 in which N=3, a first partition 34 separates the end housing 2 and the intermediate housing 3, and a second partition 36 separates the intermediate housing 3 and the other end housing 4.

The first wall 6, the second wall 8, the third wall 22, the fourth wall 24 delimit N openings giving access to the housings of the single row to allow the insertion of surgical staples into these housings 2, 3, 4 by a translational movement in the vertical direction. The partitions 34, 36 also contribute to delimiting these openings.

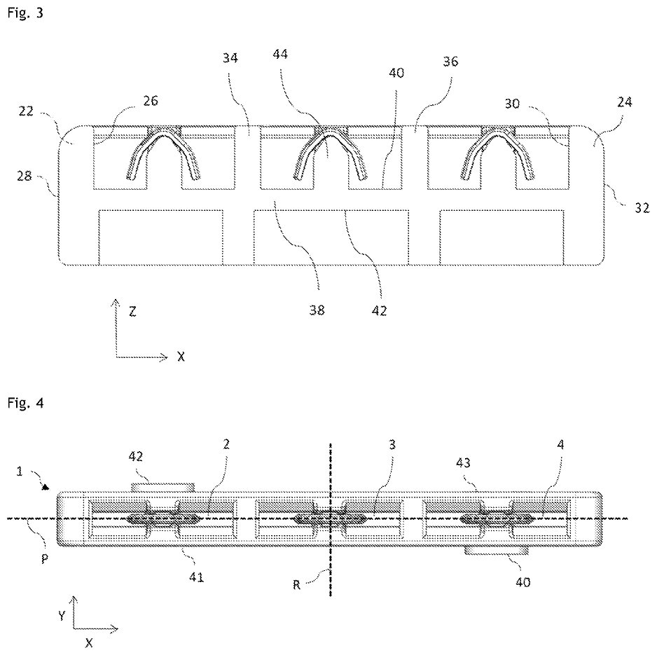

With reference to FIG. 3, cartridge 1 moreover comprises a bottom wall 38 having an upper surface 40 and a lower surface 42 opposite the upper surface 40. The upper surface 40 forms the respective bottoms of the housings 2, 3, 4, these bottoms being opposite the openings giving access respectively to the housings 2, 3, 4.

The bottom wall 38 extends in a plane parallel to the longitudinal direction and to the transverse direction.

The cartridge 1 moreover comprises supports 44, each support 44 is arranged in a housing 2, 3, 4 to be overlapped by a surgical staple, when this surgical staple is stored in the housing 2, 3, 4. Each support 44 has a convex top intended to rest against a concave surface of a surgical staple, this concave surface being formed in a connecting portion between two arms of the surgical staple.

Cartridge 1 forces surgical staples to adopt particular respective storage positions, when these staples are stored in the housings 2, 3, 4.

Each surgical staple comprises a first arm and a second arm that can rotate relative to the first arm about an axis of rotation, between an open position and a closed position. The open position, visible in FIG. 3, is a rest position of the surgical staple. In the closed position, the two arms are closer to each other than in the open position. The first arm and the second arm are connected by a connecting portion forming a pivot connection between the two arms. The relative mobility of the two arms is obtained by deformation of the connecting portion.

With reference to FIG. 4, when a staple is in the storage position in one of the housings 2, 3, 4, the surgical staple is oriented such that the axis of rotation R of the surgical staple is normal to the first inner surface 10 and to the second inner surface 14, in other words parallel to the transverse direction indicated by the axis Y.

In each housing 2, 3, 4, the transverse gap between the first wall 6 and the second wall 8, measured in the transverse direction, is chosen to force a surgical staple to adopt the preceding orientation. In particular, this transverse gap is chosen to prevent the storage of a staple in a housing in a position where the axis of rotation R of the staple would be parallel to the transverse direction, as in the cartridge 1 described in document US 2019/0133594.

Moreover, there is a reference plane P which is perpendicular to the axis of rotation R of each of the surgical staples, and which intersects each of the surgical staples, when these surgical staples are respectively stored in the housings 2, 3, 4. FIG. 3 also shows a section of the cartridge 1 in this reference plane P. In this case, the reference plane P is a median plane equidistant from the first inner surface 10 and the second inner surface 14, and which intersects each housing 2, 3, 4 of the single row of housings, as well as the third wall 22, the fourth wall 24, and each partition 34, 36.

Moreover, in its storage position, the connecting portion of a surgical staple rests on one of the supports 44, and the two arms of the staple extend on either side of the support 44 (hence the overlap mentioned above), as can be seen in FIG. 3.

By forming housings 2, 3, 4 which force the surgical staples to be disposed in the preceding positions, the width of the cartridge 1 measured along the axis Y can be greatly reduced. Moreover, the width of the cartridge 1 shown in the figures is significantly less than its length and height.

Returning to FIG. 1, the cartridge 1 comprises first blocking means 40, 41 for limiting a movement of the cartridge 1 relative to a first adjacent structure in a direction parallel to the reference plane P of the cartridge 1. It will be seen below that this adjacent structure can be another cartridge having the same features as the cartridge 1, or else a flap of a case.

More specifically, the first blocking means 40, 41 are adapted to limit a translational movement of the cartridge 1 relative to the first adjacent structure in any direction parallel to the reference plane P, in particular in the longitudinal direction and in the vertical direction.

The first blocking means comprise a first male element 40 adapted to be engaged with a female element of the first adjacent structure.

The first male element 40 projects from the first outer surface 12. The first male element 40 has a first slope extending the first planar surface, the first slope being inclined relative to the first planar surface at an obtuse angle. It will be seen below that this obtuse angle is advantageous for deflecting the trajectory of a flap sliding along the first wall 6.

The first blocking means further comprise a first female element 41 adapted to be engaged with a male element of the first adjacent structure. The first female element 41 is formed by a hole in the first outer surface 12. This hole is blind; therefore, it does not open into the first inner surface 10. The hole opens exclusively into the first outer surface 12.

The first male element 40 is arranged between the first female element 41 and the edge of the cartridge formed by the fourth wall 24. In other words, the first male element 40 is closer to the fourth wall 24 than the first female element 41. The first female element 41 is arranged between the first male element 40 and the edge formed by the third wall 22. In other words, the first female element 41 is closer to the third wall 22 than the first male element 40.

With reference to FIG. 2, cartridge 1 moreover comprises second blocking means 42, 43 for limiting a movement of the cartridge 1 relative to a second adjacent structure in a direction parallel to the reference plane of the cartridge 1.

More specifically, the second blocking means are adapted to limit a translational movement of the cartridge 1 relative to the second adjacent structure in any direction parallel to the reference plane P, in particular in the longitudinal direction and in the vertical direction. The second blocking means 42, 43 are opposite the first blocking means 40, 41 relative to the single row of housings 2, 3, 4. Thus, the first blocking means 40, 41 and the second blocking means 42, 43 are intended to cooperate respectively with two different adjacent structures between which the cartridge 1 can be placed.

The second blocking means comprise a second male element 42 adapted to be engaged with a female element of the second adjacent structure. The second male element 42 is opposite the first male element 40 relative to the single row of housings.

The second male element 42 projects from the second outer surface 16. The second male element 42 has a second slope extending the second planar surface, the second slope being inclined relative to the second planar surface at an obtuse angle. It will be seen below that this obtuse angle is advantageous for deflecting the trajectory of a flap sliding along the second wall 8.

The second blocking means further comprise a second female element 43 adapted to be engaged with a male element of the second adjacent structure. The second female element 43 is opposite the first female element 41 relative to the single row of housings. The second female element 43 is formed by a hole in the second outer surface 16. This hole is blind; therefore, it does not open into the second inner surface 14. The hole opens exclusively into the second outer surface 16.

The second male element 42 is arranged between the second female element 43 and the edge of the cartridge formed by the third wall 22. In other words, the second male element 42 is closer to the third wall 22 than the second female element 43. The second female element 43 is arranged between the second male element 42 and the edge formed by the fourth wall 24. In other words, the second female element 43 is closer to the fourth wall 24 than the second male element 42.

The second blocking means 42, 43 are symmetrical to the first blocking means 40, 41 relative to a central axis of the cartridge 1 parallel to the vertical direction. In particular, the second male element 42 is symmetrical to the first male element 40 relative to this central axis, and the second female element 43 is symmetrical to the first female element 41 relative to this central axis.

### 2) Set of Cartridges

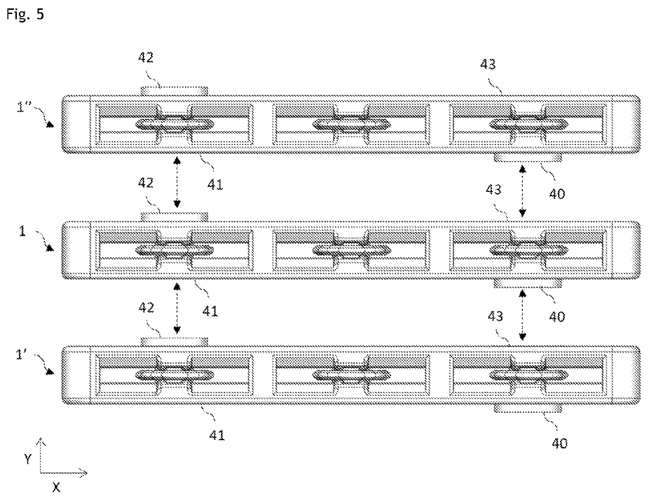

FIG. 5 shows a set of cartridges comprising cartridge 1.

The set of cartridges moreover comprises a first adjacent cartridge 1′ acting as a first adjacent structure for the cartridge 1. Thus, the first blocking means 40, 41 of the cartridge 1 are adapted to limit movement of the cartridge 1 relative to the first adjacent cartridge 1′ in a direction parallel to the reference plane of the cartridge 1.

The first adjacent cartridge 1′ has the same structure as cartridge 1.

A limitation of movement of the cartridge 1 relative to the first adjacent cartridge 1′ in a direction parallel to the reference plane of the cartridge (and parallel to the reference plane of the first adjacent cartridge 1′) can be achieved by stacking the cartridge 1 and the first adjacent cartridge 1′ in each of the following alternative positions:
In a first position shown in FIG. 5, in which the first wall 6 of the cartridge 1 is positioned against the second wall 8 of the first adjacent cartridge 1′ and in which the first blocking means 40, 41 of cartridge 1 are engaged with the second blocking means 42, 43 of the first adjacent cartridge 1′.
In a second position, in which the first wall 6 of the cartridge 1 is positioned against the first wall 6 of the first adjacent cartridge 1′, and in which the first blocking means 40, 41 of cartridge 1 are engaged with the first blocking means 40, 41 of the first adjacent cartridge 1′.
In a third position, in which the second wall 8 of the cartridge 1 is positioned against the first wall 6 of the first adjacent cartridge 1′, and in which the second blocking means 42, 43 of cartridge 1 are engaged with the first blocking means 40, 41 of the first adjacent cartridge 1′,
In a fourth position, in which the second wall 8 of cartridge 1 is positioned against the second wall 8 of the first adjacent cartridge 1′, and in which the second blocking means 42, 43 of cartridge 1 are engaged with the second blocking means 42, 43 of the first adjacent cartridge 1′.

In each of these positions, a male element (40 or 42) of the cartridge 1 is engaged with a female element (41 or 43) of the first adjacent cartridge 1′, and a female element (41 or 43) of the cartridge 1 is engaged with a male element (40 or 42) of the first adjacent cartridge 1′.

It should be noted that the symmetrical nature of the first and second blocking means described above contributes to making these multiple alternative positions possible. However, these alternative positions could also be obtained with blocking means which are not perfectly symmetrical in the cartridge 1 and/or in the first adjacent cartridge 1′.

The set of cartridges further comprises a second adjacent cartridge 1″ acting as a second structure adjacent to the cartridge 1, such that the cartridge 1 is located between the first adjacent cartridge 1′ and the second adjacent cartridge 1″.

The second adjacent cartridge 1″ has a shape identical to that of the cartridge 1, which allows mutual locking between the cartridge and the second adjacent cartridge 1″ by placing the cartridge and the second cartridge in four different alternative positions, as already described above for the cartridge and the first adjacent cartridge 1′.

### 3) Case for a Set of Cartridges

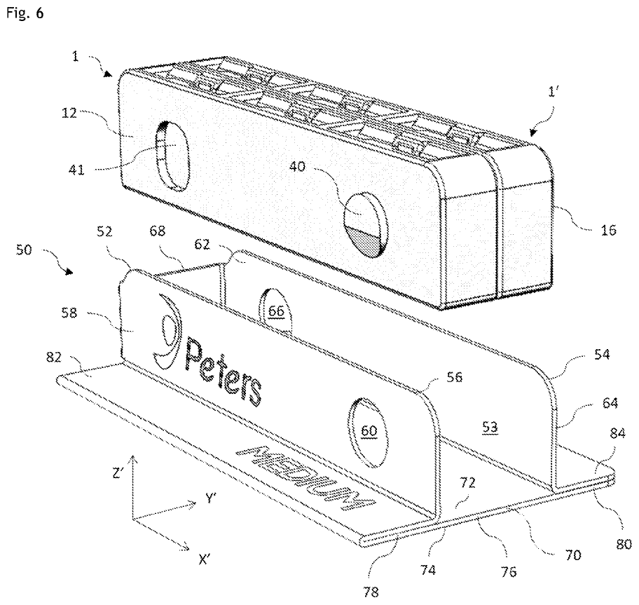

With reference to FIG. 6, a case 50 has the function of holding the cartridges of a set of stacked cartridges.

The case 50 extends in a longitudinal direction indicated by an axis X′, in a transverse direction indicated by an axis Y′, and a vertical direction indicated by an axis Z′, the axes X′, Y′ and Z′ forming an orthogonal reference frame.

Generally, the case has an elongated shape in the longitudinal direction, in the sense that its length measured parallel to the axis X′ is greater than its width measured parallel to the axis Y′ and its height measured parallel to the axis Z′.

The case comprises a first flap 52 and a second flap 54 at a distance from the first flap 52, so as to define therebetween a space 53 for storing a set of K identical cartridges. For this purpose, the distance between the flaps 52, 54 is substantially equal to K times the width of one of the cartridges. In the embodiment illustrated in FIG. 6, the set of cartridges consists of cartridges 1 and 1′ (K=2), but in other embodiments the set of cartridges may comprise a greater number of identical cartridges (for example the set formed by the three cartridges 1, 1′ and 1″ shown in FIG. 5, or a set comprising more cartridges).

The first flap 52 has a first inner surface 56 delimiting the space 53, and a first outer surface 58 opposite the first inner surface 56. The first flap 52, in particular the first inner surface 56, extends in the longitudinal direction indicated by the axis X′.

The first flap 52 comprises a first female element 60. The first female element 60 is in the shape of a hole opening into the first inner surface 56. The female element 60 also opens into the first outer surface 58; in other words, the female element 60 is formed by a through hole.

The second flap 54 has a second inner surface 62 delimiting the space 53, and a second outer surface 64 opposite the second inner surface 62. The second inner surface 62 faces the first inner surface 56. The second flap 54, in particular the second inner surface 62, extends in the longitudinal direction indicated by the axis X′.

The second flap 54 comprises a second female element 66. The second female element 66 is in the shape of a hole opening into the second inner surface 62. The female element also opens into the second outer surface 64; in other words, the female element 66 is formed by a through hole.

The case 50 moreover comprises a connecting flap 68 between the first flap 52 and the second flap 54. The connecting flap 68 is connected to the first flap 52 by a first vertical fold line, and is connected to the second flap 54 by a second vertical fold line. The first vertical fold line and the second vertical fold line are parallel to the vertical axis Z′.

The case 50 further comprises a base 70 intended to stabilize the case 50 when the case 50 is placed on a support.

The case 50 moreover defines an access to the space 53 via which the set of cartridges 1, 1′ can be inserted. The access comprises an upper opening opposite the base 70 relative to space 53, and a lateral opening opposite the connecting flap 68 relative to space 53. In this way, the set of cartridges can be inserted into space 53 via the upper opening, or via the lateral opening, according to orthogonal translations.

The first flap 52, the second flap 54 and the connecting flap 68 extend transversely to the base 70.

The base 70 has an upper surface 72 and a lower surface 74 opposite the upper surface 72. The lower surface 74 is intended to rest on a support. The lower surface 72 is flat.

The base 70 comprises a central portion 76 forming a bottom of the space 53, and extending from the first flap 52 to the second flap 54.

The connecting flap 68 is connected to the central portion 76 by a third fold line, perpendicular to the first fold line and the second fold line.

The base 70 further comprises a first peripheral portion 78, and a second peripheral portion 80 opposite the first peripheral portion 78 relative to the central portion 76. In other words, the central portion 76 extends between the first peripheral portion 78 and the second peripheral portion 80. The first peripheral portion 78 extends the central portion 76 beyond the first flap 52, and the second peripheral portion 80 extends the central portion 76 beyond the second flap 54. The central portion 76, the first peripheral portion 78 and the second peripheral portion 80 are coplanar.

The two peripheral portions 78 and 80 have the advantage of giving the case 50 greater stability. They prevent the case from tilting by rotation around an axis parallel to the axis X′.

The base 70 further comprises an additional connecting flap 82 connecting the first flap 52 to the first peripheral portion 78. The connecting flap 82 is connected to the first peripheral portion 78 by a third longitudinal fold line. The connecting flap 82 is folded against the upper surface 72 of the base 70, at the first peripheral portion 78. The connecting flap 82 is further connected to the first flap 52 by a fourth longitudinal fold line. The third longitudinal fold line and the fourth longitudinal fold line are parallel to the axis X′.

The base 70 further comprises another additional connecting flap 84 connecting the second flap 54 to the second peripheral portion 80. The connecting flap 84 is connected to the second flap 54 by a fifth longitudinal fold line parallel to the axis X′. Furthermore, the connecting flap 84 is glued to the upper surface 72, against the second peripheral portion 80.

The case 50 is formed from a single piece, made of a deformable material, typically cardboard. This piece of cardboard is folded on itself at the longitudinal and vertical fold lines mentioned above, and the flaps 82 and 84 are glued so as to form case 50.

The first flap 52 is movable between several positions. A first position of the first flap 52, shown in FIG. 6, constitutes a rest position of the first flap, that is to say the position that the first flap 52 naturally takes in the absence of external stress. When the first flap 52 is in its first position, the first inner surface 56 is planar, and extends in the vertical direction indicated by the axis Z′, that is to say perpendicular to the base 70.

The first flap 52 may further be placed in a second position by pressing on the first inner surface 56, causing a deformation of the first flap 52 and partially separating the first flap 52 from the second flap 54. In the second position, the first inner surface 56 is convex, while the first outer surface 58 is concave. Moreover, the female element 60 is further from the second flap 54 in the second position than in the first position. In the second position of the first flap 52, the first flap 52 remains connected to the flaps 68 and 82 via the first vertical fold line and the first longitudinal fold line, respectively.

Similarly, the second flap 54 is also movable between several positions.

A first position of the second flap 54, shown in FIG. 6, constitutes a rest position of the second flap 54, that is to say the position that the second flap 54 naturally takes in the absence of external stress. When the second flap 54 is in its first position, the second inner surface 62 is planar, and extends in the vertical direction indicated by the axis Z′, that is to say perpendicular to the base 70.

The second flap 54 may further be placed in a second position by pressing on the second inner surface 62, causing a deformation of the second flap 54 and partially separating the second flap 54 from the first flap 52. In the second position, the second inner surface 62 is convex, while the second outer surface 64 is concave. Furthermore, the female element 66 is further from the first flap 52 in the second position of the second flap 54 than in its first position. In the second position of the second flap 54, the second flap 54 remains connected to the flaps 68 and 84 via the second vertical fold line and the second longitudinal fold line, respectively.

### 4) Method for Packaging Cartridges

A method for packaging surgical staples comprises the following steps.

A user places surgical staples in the housings 2, 3, 4 of cartridge 1, and places surgical staples in the housings 2, 3, 4 of the cartridge 1′, as illustrated in FIGS. 1 to 4.

Moreover, the user stacks cartridges 1 and 1′ by pressing the second outer surface 16 of the cartridge 1 against the first outer surface 12 of the cartridge 1′, so that the second blocking means 42, 43 of the cartridge 1 are engaged with the first blocking means 40, 41 of the cartridge 1′.

The first outer surface 12 of cartridge 1 (on the left in FIG. 6) and the second outer surface 16 of the cartridge 1′ (on the right in FIG. 6) form two opposite edges of the set of cartridges thus formed. It should be noted that the width of the set formed by the stacked cartridges 1, 1′, measured between these two opposite edges, is substantially equal to the width of the space 53 defined by the case 50, that is to say the distance which separates the first flap 52 from the second flap 54 measured parallel to the axis Y′.

The case 50 is placed on a horizontal support, with the lower surface 76 of the base 70 facing downwards, in contact with the horizontal support.

A user grasps the set of cartridges 1, 1′ by its two opposite edges formed by the first outer surface 12 of the cartridge 1 and by the second outer surface 16 of the cartridge 1′, so as to keep the cartridges 1 and 1′ stacked. The user positions the set of cartridges 1, 1′ above space 53, in an orientation such that the axes X and X′ are parallel, the axes Y and Y′ are parallel and the axes Z and Z′ are parallel, as shown in FIG. 6. The openings of the housings 2, 3, 4 of the cartridges 1 and 1′ are oriented upwards. Moreover, the user substantially aligns the first outer surface 12 of cartridge 1 with the inner surface 56 of the first flap, and substantially aligns the second outer surface 16 with the inner surface 62 of the second flap 54.

The user inserts the set of cartridges 1, 1′ into space 53 by translating it in the vertical direction indicated by the axis Z′, towards the bottom of the space 53 formed by the base 70.

During this insertion, the first outer surface 12 of the cartridge 1 runs along the inner surface 56 of the first flap 52, or even slides against the latter. At a certain stage, the first flap 52 bears on the inclined slope of the male element 40 of the cartridge 1. However, the insertion can continue due to the obtuse nature of the angle of the inclined slope. The sliding of the first flap 52 against the inclined slope of the male element 40 moves the first flap from its first position (rest position) to its second position discussed previously. When the set of cartridges 1, 1′ abuts against the base 70, the first male element 40 of the cartridge 1 is aligned with the hole formed by the female element 60, so that the first male element 40 no longer presses on the first flap 52. At this stage, the first flap 52 returns to its first position by elastic return, and the first male element 40 of the cartridge 1 then engages in the hole formed by the female element 60. The first male element 40 of the cartridge 1 and the female element 60 are thus engaged, which has the effect of limiting or even preventing a translational movement of the cartridge 1 relative to the first flap 52 along the axis X′ and along the axis Z′, and therefore an exit of the set of cartridges from the space 53.

Moreover, during insertion, the second outer surface 16 of the cartridge 1′ runs along the inner surface 62 of the second flap 54, or even slides against the latter. At a certain stage, the second flap 54 bears on the inclined slope of the male element 42 of the cartridge 1′. The sliding of the second flap 54 against the inclined slope of the male element 42 moves the second flap 54 from its first position (rest position) to its second position. When the set of cartridges 1, 1′ abuts against the base 70, the second male element 42 of the cartridge 1′ is aligned with the hole formed by the female element 66, so that the second male element 42 of the cartridge 1′ no longer presses on the second flap 54. At this stage, the second flap 54 returns to its first position by elastic return, and the second male element 42 of the cartridge 1′ then engages in the hole formed by the female element 66. The second male element 42 of the cartridge 1 and the female element 66 are thus engaged, which has the effect of limiting or even preventing a translational movement of the cartridge 1′ relative to the second flap 54 along the axis X′ and along the axis Z′, and therefore an exit of the set of cartridges from the space 53.

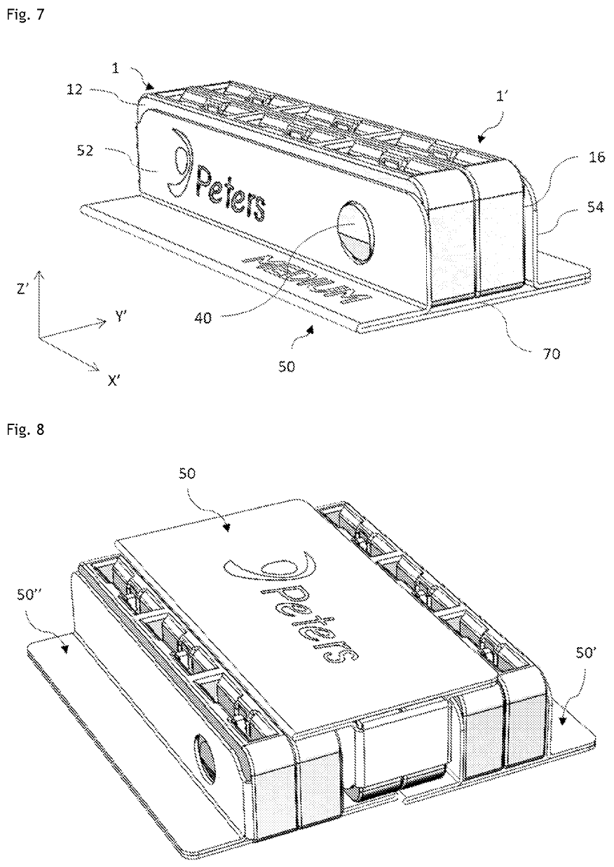

The set of cartridges is now stored in space 53 of the case 50, in a position shown in FIG. 7 where the cartridges 1 and 1′ are kept stacked and where the respective reference planes of the cartridges 1 and 1′ extend parallel to the first flap 52 and the second flap 54.

It is also noted that the two flaps 52 and 54 limit or even prevent translational movement of the set of cartridges 1, 1′ relative to the case 50 along the axis Y′.

The inclined slopes formed on the male elements 40 and 42 of the cartridges 1 and 1′ have the advantage of facilitating the insertion of the set of cartridges 1, 1′ into the space 53. Thanks to these slopes, the user does not need to manually separate the two flaps 52 and 54 from each other.

It is also possible to insert the set of cartridges 1, 1′ into space 53 via a translational movement along the axis X′, after having placed the set of cartridges opposite the connecting flap 68. In this case, the user must manually separate the two flaps 52, 54.

With reference to FIG. 8, the preceding steps are repeated for other cases 50′ and 50″ having the same features as the case 50. Then, the cases 50, 50′ and 50″ can be stacked head to tail so as to optimize the storage space of the set of cases 50, 50′, 50″.

The above method can be generalized to other embodiments in which the number K of cartridges in the set is greater than or equal to three. In these embodiments, there is at least one cartridge which is surrounded by two other cartridges in the set of cartridges, as is the case with the cartridge 1 in the set of three cartridges shown in FIG. 5. Each cartridge of this type is not intended to be engaged with case 50, but to be engaged with the two adjacent cartridges which surround it, which allows to maintain the set in the space 53 of the case 50.

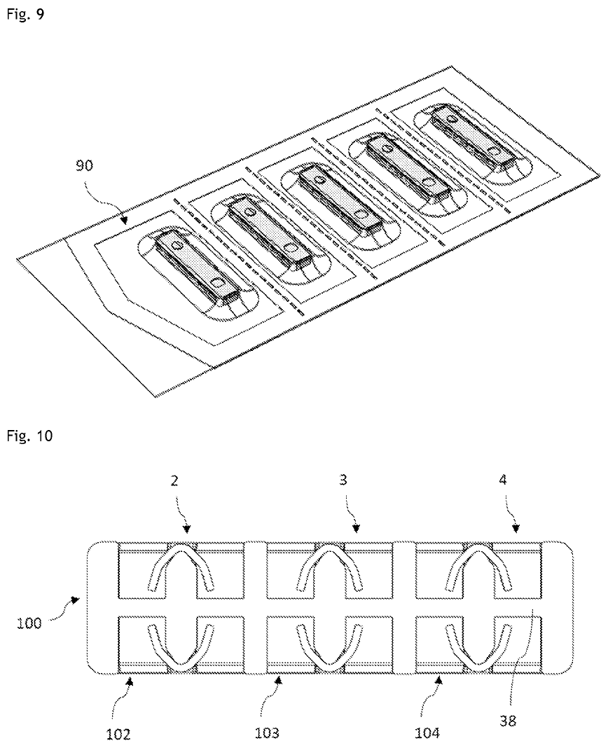

Another type of packaging is shown in FIG. 9. The packaging 90 such as a peelable bag contains a plurality of cartridges 1. The cartridges 1 are disposed in the packaging 90 so that their respective planes P coincide. Thanks to this arrangement, the packaging 90 can remain very flat.

### 5) Use of the Cartridge Intracorporeally

Cartridge 1 is very well suited to intracorporeal use. Such use typically involves the following steps.

Cartridge 1 is placed between the jaws of a first instrument such as a clamp, such that each housing of the cartridge remains accessible from the outside by one of the two opposite sides of the jaws. For this purpose, the cartridge is oriented such that: one of the jaws rests on the first outer surface 12 and the other jaw rests on the second outer surface 16.

The jaws of the first instrument may be movable (such as the jaws of a clamp) or fixed and parallel to the outer surfaces 12 and 16, such that the space between the two jaws is less than the distance between the first outer surface 12 and the second outer surface 16.

The first instrument is then moved through a first trocar introduced into a patient's body, towards a placement site for at least one of the staples stored in the cartridge 1. The elongated shape of the cartridge 1 allows it to be inserted into a trocar of reduced diameter, which is very advantageous.

An applicator suitable for removing surgical staples, constituting a second instrument, is placed in a second trocar introduced into the patient's body.

From outside the patient's body, a user can move the applicator inside the body relative to the first instrument, so as to grasp and pick up a staple from the cartridge 1 held between the jaws of the first instrument, and bring the grasped staple to the site where this staple is to be placed. These steps can be repeated to place several staples stored in cartridge 1 at the placement site. As indicated in the introduction, this method avoids the user having to go back and forth between the inside and outside of the body to place different staples and thus reduces the duration of the operation.

### 6) Other Embodiments

FIG. 10 shows a cartridge 100 according to a second embodiment. Cartridge 100 comprises all the walls, partitions and blocking means of the cartridge 1 described above.

The cartridge 100 differs from the cartridge 1 in that it comprises not a single row of housings, but two rows of housings: a first row of N housings opening onto a first side of the cartridge 100 (corresponding to the single row of housings of the cartridge 1), and a second row of N housings 102, 103, 104 opening onto a second side of the cartridge opposite the first side of the cartridge 100.

Just as in cartridge 1, there is a reference plane P which is perpendicular to the axis of rotation R of each of the surgical staples, and which intersects each of the surgical staples, when these surgical staples are respectively stored in the housings of the first row and the second row of the cartridge 100. FIG. 10 also shows a section of the cartridge 100 in this reference plane P.

The bottom wall 38 forms not only the respective bottoms of the housings 2, 3, 4 of the first row, but also the respective bottoms of the housings 102, 103, 104 of the second row. In other words, the bottom wall 38 separates the first row of housings and the second row of housings.

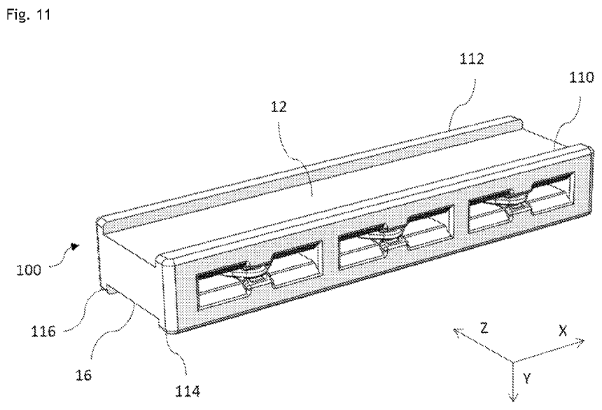

With reference to FIG. 11, the cartridge 100 moreover comprises two first stops 110, 112 projecting from the outer surface 12 of the first wall 6, to delimit therebetween a first recess for receiving a first jaw of the first instrument as mentioned previously. The stop 110 runs along the openings of the housings of the first row of housings, while the stop 112 runs along the openings of the housings of the second row of housings. The stops 110, 112 extend rectilinearly in the longitudinal direction parallel to the axis X.

The cartridge 100 further comprises two second stops 114, 116 projecting from the outer surface 16 of the second wall 8, so as to delimit therebetween a second recess for receiving a second jaw of the first instrument as mentioned previously. The stop 114 runs along the openings of the housings of the first row of housings, while the stop 116 runs along the openings of the housings of the second row of housings. The stops 114, 116 extend rectilinearly in the longitudinal direction parallel to the axis X.

Cartridge 100 can of course constitute an alternative to cartridge 1, 1′ or 1″ in all the uses as described previously.

In particular, the cartridge 100 has the advantage of being even easier to use intracorporeally. The first jaw is received in the recess formed between the first stops 110, 112, and the second jaw is received in the recess formed between the second stops 114, 116. The first jaw bears on the first outer surface 12 at the bottom of the first recess, and the second jaw bears on the second outer surface 16 at the bottom of the second recess.

In this grasped position, the housings of the first row are accessible by a first lateral side of the first instrument and the housings of the second row are accessible by a second lateral side of the first instrument opposite the first lateral side.

In this grasped position, the stops 110, 112, 114, 116 limit or even prevent a movement of the cartridge 100 in a direction transverse to the jaws, thus preventing the cartridge from escaping through one of the two lateral sides of the first instrument.

A user wishing to remove a staple from the cartridge 100, while the latter is full, can choose to orient the first instrument in two different ways to allow the extraction of a staple contained in a housing of the first row, or of a staple contained in a housing of the second row.

1. A cartridge comprising compartments for storing respective surgical staples, each of the surgical staples comprising a first arm and a second arm rotatably movable about an axis of rotation relative to the first arm in order to close the surgical staple, wherein all the compartments of the cartridge are designed so that, when the surgical staples are respectively stored in all the compartments of the cartridge, the surgical staples adopt respective positions such that a plane perpendicular to the axis of rotation of each of the surgical staples intersects each of the surgical staples.

2. The cartridge of claim 1 comprising the surgical staples respectively stored in all the compartments.

3. The cartridge of claim 1, wherein the compartments comprise a first row of compartments opening onto a first side of the cartridge, and a second row of compartments opening onto a second side of the cartridge opposite the first side.

4. The cartridge of claim 1, comprising:
a first wall and a second wall delimiting the compartments therebetween, the first wall having a first outer surface and the second wall having a second outer surface opposite the first outer surface,
two stops projecting from the first outer surface to delimit therebetween a recess, the two stops being arranged to allow the cartridge to be placed in a position gripped by an instrument wherein:
a first jaw of the instrument bears on the first outer surface and a second jaw of the instrument bears on the second outer surface,
each housing of the cartridge is accessible via a first side of the instrument or via a second side of the instrument opposite the first side,
the first jaw extends into the recess between the two stops.

5. The cartridge of claim 4, comprising two second stops projecting from the second outer surface to delimit therebetween a second recess, the two second stops being arranged so that, in the gripped position, the second jaw extends into the second recess between the second stops.

6. The cartridge of claim 1, comprising first blocking means for limiting a movement of the cartridge relative to a first adjacent structure in a direction parallel to the plane, the first blocking means comprising at least one of:
a first male element adapted to be engaged with a female element of the adjacent first structure,
a first female element adapted to be engaged with a male element of the adjacent first structure.

7. The cartridge of claim 6, wherein the first blocking means are adapted to limit a translational movement of the cartridge relative to the first adjacent structure in any direction parallel to the plane.

8. The cartridge of claim 6, comprising a first wall having a first planar surface, wherein the first blocking means comprise the first male element, and wherein the first male element projects from the first planar surface and has a first slope extending the first planar surface, the first slope being inclined relative to the first planar surface at an obtuse angle.

9. The cartridge of claim 6, comprising second blocking means for limiting movement of the cartridge relative to a second adjacent structure in a direction parallel to the plane, the second blocking means being opposite the first blocking means relative to the compartments the second blocking means comprising at least one of:
a second male element adapted to be engaged with a female element of the second adjacent structure,
a second female element adapted to be engaged with a male element of the second adjacent structure.

10. The cartridge of claim 9, wherein the second blocking means are adapted to limit a translational movement of the cartridge relative to the second adjacent structure in any direction parallel to the plane.

11. The cartridge of claim 9, wherein the second blocking means are symmetrical to the first blocking means relative to an axis.

12. A set of cartridges comprising:
a first cartridge according to claim 9,
a second cartridge according to claim 6,
wherein the first blocking means of the second cartridge are adapted to:
be engaged with the first blocking means of the first cartridge when the first cartridge is in a first orientation relative to the second cartridge, and
be engaged with the second blocking means of the first cartridge when the first cartridge is in a second orientation relative to the second cartridge, the second orientation being different from the first orientation.

13. A system comprising:
a set of cartridges comprising:
a first cartridge according to claim 1,
a second cartridge according to claim 1,
a case comprising:
a first flap extending in a first plane and a second flap extending in a second plane, the first flap and the second flap defining therebetween a space for storing the set of cartridges in a stacked position in which the plane of the first cartridge and the plane of the second cartridge are parallel to the first plane and the second plane.

14. The system of claim 13, wherein the first flap is movable, while the set of cartridges is stored in the space in the stacked position, between:
a first position in which the first flap is engaged with the first cartridge, to limit a translational movement of the first cartridge relative to the first flap in a direction parallel to the first plane,
a second position, in which the first flap is not engaged with the first cartridge.

15. The system of claim 14, wherein:
the case defines an access to the space allowing insertion of the set of cartridges into the space,
the first cartridge is in accordance with claim 8,
the first slope is arranged so that, during insertion of the set of cartridges into the space via the access, the first flap bears on the slope, and sliding of the first flap on the slope moves the first flap from the first position to the second position.

16. The system of claim 14, wherein
the first flap comprises a female element,
the first male element of the first cartridge engages with the female element of the first flap in the first position.

17. The system of claim 13, wherein the second flap is movable, while the set of cartridges is stored in the space in the stacked position, between:
a first position in which the second flap is engaged with the second cartridge to limit a translational movement of the second cartridge relative to the second flap in a direction parallel to the second plane,
a second position, in which the second flap is not engaged with the second cartridge.

18. The system of claim 13, wherein:
the case defines an access to the space allowing insertion of the set of cartridges into the space,
the case comprises a base comprising:
a central portion forming a bottom of the space opposite the access,
a first peripheral portion extending the central portion 76 beyond the first flap, and optionally a second peripheral portion extending the central portion beyond the second flap, the second peripheral portion being opposite the first peripheral portion relative to the central portion.

19. The system of claim 13, wherein:
the case defines an access to the space allowing the set of cartridges to be inserted into the space,
the case comprises:
a base forming a bottom of the space opposite the access, the first flap being connected to the base by a first fold line, and the second flap being connected to the base by a second fold line,
a connecting flap connected to the first flap by a third fold line perpendicular to the first fold line and connected to the second flap by a fourth fold line perpendicular to the second fold line.

20. The system claim 13, wherein the case is made of cardboard.
